(19) United States
(12) Patent Application Publication
(10) Pub. No.: US 2010/0305023 A1
(43) Pub. Date: Dec. 2, 2010

(54) Method of Delaying The Onset of Clinically Definite Multiple Sclerosis

(75) Inventors: Yafit Stark, Giv'at Shmu'el (IL); David Ladkani, Jerusalem (IL)

Correspondence Address: COOPER & DUNHAM, LLP, 30 Rockefeller Plaza, 20th Floor, NEW YORK, NY, 10112, US

(73) Assignee: Teva Pharmaceutical Industries, Ltd.

(21) Appl. No.: 12/861,655

(22) Filed: Aug. 23, 2010

Related U.S. Application Data
(63) Continuation of application No. 12/315,009, filed on Nov. 26, 2008.
(60) Provisional application No. 61/004,710, filed on Nov. 28, 2007.
(60) Provisional application No. 61/005,271, filed on Dec. 3, 2007.
(60) Provisional application No. 61/007,141, filed on Dec. 11, 2007.
(60) Provisional application No. 61/192,455, filed on Sep. 17, 2008.

(51) Int. Cl. A61K 38/02; A61P 37/00

(52) U.S. Cl. 514/1.1

(57) ABSTRACT

A method for delaying the onset of clinically definite multiple sclerosis in a patient at risk of developing clinically definite multiple sclerosis and retard long-term progression of multiple sclerosis and its symptoms, the method comprising periodically administering a pharmaceutical composition comprising a therapeutically effective amount of glatiramer acetate to the patient, thereby delaying onset of clinically definite multiple sclerosis in the patient and retarding long-term progression of multiple sclerosis and its symptoms.

This application is a continuation of U.S. Ser. No. 12/315,009, filed Nov. 26, 2008, which claims the benefits of U.S. Provisional Application Nos. 61/004,710, filed Nov. 28, 2007, 61/005,271, filed Dec. 3, 2007, 61/007,141, filed Dec. 11, 2007 and 61/192,455, filed Sep. 17, 2008, the contents of which are hereby incorporated by reference in their entirety.

Throughout this application various publications are referenced by Arabic numeral in parentheses. The full citation of the corresponding reference appears at the end of the specifications before the claims. The disclosures of these publications in their entireties are hereby incorporated by reference into this application in order to more fully describe the state of the art to which this invention pertains.

### BACKGROUND OF THE INVENTION

With over 2 million afflicted people worldwide, multiple sclerosis (“MS”) is one of the more common chronic neurological diseases in human adults. MS is a chronic, inflammatory central nervous system (CNS) disease characterized pathologically by demyelination. MS has also been classified as an autoimmune disease.

MS disease activity can be monitored by cranial scans, including magnetic resonance imaging (MRI) of the brain, accumulation of disability, as well as rate and severity of relapses. The diagnosis of clinically definite MS as determined by the Poser criteria (1) requires at least two neurological events suggesting demyelination in the CNS separated in time and in location. A clinically isolated syndrome (CIS) is a single monosymptomatic attack suggestive of MS, such as optic neuritis, brain stem symptoms, and partial myelitis. Patients with CIS that experience a second clinical attack are generally considered to have clinically definite multiple sclerosis (CDMS). Over BO percent of patients with a CIS and MRI lesions go on to develop MS, while approximately 20 percent have a self-limited process (2, 3).

There are five distinct disease stages and/or types of MS:

1) benign multiple sclerosis;
2) relapsing-remitting multiple sclerosis (RRMS);
3) secondary progressive multiple sclerosis (SPMS);
4) progressive relapsing multiple sclerosis (PRMS); and
5) primary progressive multiple sclerosis (PPMS)

Benign multiple sclerosis is a retrospective diagnosis which is characterized by 1-2 exacerbations with complete recovery, no lasting disability and no disease progression for 10-15 years after the initial onset. Benign multiple sclerosis may, however, progress into other forms of multiple sclerosis.

Patients suffering from RRMS experience sporadic exacerbations or relapses, as well as periods of remission. Lesions and evidence of axonal loss may or may not be visible on MRI for patients with RRMS.

SPMS may evolve from RRMS. Patients afflicted with SPMS have relapses, a diminishing degree of recovery during remissions, less frequent remissions and more pronounced neurological deficits than RRMS patients. Enlarged ventricles, which are markers for atrophy of the corpus callosum, midline center and spinal cord, are visible on MRI of patients with SPMS.

PPMS is characterized by a steady progression of increasing neurological deficits without distinct attacks or remissions. Cerebral lesions, diffuse spinal cord damage and evidence of axonal loss are evident on the MRI of patients with PPMS. PRMS has periods of acute exacerbations while proceeding along a course of increasing neurological deficits without remissions. Lesions are evident on MRI of patients suffering from PRMS (5).

Glatiramer acetate (GA), a mixture of polypeptides which do not all have the same amino acid sequence, is marketed under the trade name Copaxone®. GA comprises the acetate salts of polypeptides containing L-glutamic acid, L-alanine, L-tyrosine and L-lysine at average molar fractions of 0.141, 0.427, 0.095 and 0.338, respectively. The average molecular weight of Copaxone® is between 5,000 and 9,000 daltons (6). Chemically, glatiramer acetate is designated L-glutamic acid polymer with L-alanine, L-lysine, L-tyrosine, acetate (salt). Its structural formula is:

(Glu,Ala,Lys,Tyr)ₓ.xCH₃COOH

(C₅H₉NO₄.C₃H₇NO₂.C₆H₁₄N₂O₂.C₉H₁₁NO₃)ₓ.xCHO

CAS-147245-92-9

Copaxone® (20 mg glatiramer acetate injection) is an approved therapy for patients with RRMS. The synthesis of Copaxone® has been disclosed, for example, in U.S. Pat. Nos. 3,849,550, 6,939,539, 5,800,808 and 7,199,098. The formulation of 40 mg Copaxone® has been disclosed in US Patent Publication No. US2007/0161566. The entire contents of these publications are hereby incorporated by reference.

The efficacy of Copaxone® in reducing the frequency of relapses in patients with RRMS is well established (7,8). The 20 and 40 mg/day subcutaneous dose has been shown to reduce the total number of enhancing lesions in MS patients as measured by MRI (8,9). However, it is an open question whether Copaxone® therapy would be effective in subjects suffering from earlier stages of MS. Moreover, a debate exists in the medical and scientific communities as to the benefit of commencing MS therapy at an early stage. Specifically, questions exist regarding whether the benefits of early treatment outweigh the inconvenience, cost, potential adverse effects of treatment, and the risk of submitting patients that independently of treatment would not experience further events to unnecessary long-term therapy (10, 11 and 12).

### SUMMARY OF THE INVENTION

This invention provides a method for reducing the frequency of relapses in a patient who experienced a single clinical attack consistent with multiple sclerosis and who has at least one lesion consistent with multiple sclerosis prior to development of clinically definite multiple sclerosis (CDMS), the method consisting essentially of periodically administering to the patient a pharmaceutical composition comprising a pharmaceutically acceptable carrier and an amount of glatiramer acetate therapeutically effective to increase the time to relapse in the patient.

This invention also provides a method for delaying onset of clinically definite multiple sclerosis in a patient presenting a first clinical event consistent with multiple sclerosis and at least one lesion consistent with multiple sclerosis comprising periodically administering to the patient as monotherapy a pharmaceutical composition comprising a pharmaceutically acceptable carrier and an amount of glatiramer acetate therapeutically effective to delay onset of clinically definite multiple sclerosis.

This invention further provides a method of treating a patient who has experienced a first clinical episode and has MRI features consistent with multiple sclerosis (MS) consisting essentially of subcutaneously administering once a day to the patient prior to conversion to clinically definite multiple sclerosis (CDMS) a pharmaceutical composition comprising mannitol and 20 mg of glatiramer acetate.

This invention yet further provides a method for delaying the onset of clinically definite multiple sclerosis in a patient at risk of developing clinically definite multiple sclerosis, the method comprising periodically administering a pharmaceutical composition comprising a therapeutically effective amount of glatiramer acetate to the patient, thereby delaying onset of clinically definite multiple sclerosis in the patient.

This invention yet further provides a method for reducing progression of magnetic resonance imaging (MRI)-monitored disease activity in a patient at risk for developing clinically definite multiple sclerosis, the method comprising periodically administering a pharmaceutical composition comprising a therapeutically effective amount of glatiramer acetate to the patient thereby reducing progression of MRI-monitored disease activity in the patient.

This invention yet further provides a method for reducing the progression of symptoms of Multiple Sclerosis in a patient, the method comprising periodically administering a pharmaceutical composition comprising a therapeutically effective amount of glatiramer acetate to the patient prior to development of clinically definite multiple sclerosis in the patient, thereby reducing the progression of symptoms of MS in the patient.

This invention yet further provides a method for reducing the frequency of relapse in a patient who experienced a single clinical attack consistent with multiple sclerosis and who has at least one lesion consistent with multiple sclerosis comprising periodically administering to the patient a pharmaceutical composition comprising an amount of glatiramer acetate therapeutically effective to increase the time to relapse in the patient.

This invention yet further provides a method for delaying progression to clinically definite multiple sclerosis in a patient presenting a first clinical event suggestive of multiple sclerosis and at least one lesion of multiple sclerosis comprising periodically administering to the patient a pharmaceutical composition comprising an amount of glatiramer acetate therapeutically effective to delay progression to clinically definite multiple sclerosis.

This invention yet further provides use of glatiramer acetate in the manufacture of a medicament for delaying the onset of clinically definite multiple sclerosis, for reducing progression of magnetic resonance imaging (MRI)-monitored disease activity, or reducing progression of symptoms of multiple sclerosis in a patient at risk for developing clinically definite multiple sclerosis.

This invention additionally provides use of glatiramer acetate in the manufacture of a medicament for the treatment of a patient who experienced a single demyelinating event and an active inflammatory process, which are indicative of the patient being at high risk of developing clinically definite multiple sclerosis.

This invention further provides glatiramer acetate for use in treating of a patient who experienced a first clinical event suggestive of multiple sclerosis and is at risk of developing clinically definitive multiple sclerosis.

This invention yet further provides use of glatiramer acetate in the manufacture of a medicament for the treatment of a patient who experienced a first clinical event suggestive of multiple sclerosis and is at risk of developing clinically definite multiple sclerosis.

### BRIEF DESCRIPTION OF FIGURES

FIG. 1 shows the time to conversion to CDMS, based on Kaplan-Meier analysis. Considering the 25^{th} percentile, glatiramer acetate prolonged the time to conversion to CDMS from 336 days on placebo to 722 days, reflecting more than twofold prolongation in slowing the onset of CDMS.

FIG. 2 shows the Kaplan-Meier survival curves and log rank test by an alternative analysis to the Cox Model in case that the proportional hazards assumption is violated.

FIG. 3 shows the total number of new T2 lesions when examined at the last observed value (LOV).

FIG. 4 shows the total number of new T2 lesions when compared annually.

FIG. 5 shows the total number of new T2 lesions in the ITT cohort when compared annually.

FIG. 6 shows the total number of new T1 Gd-enhancing lesions when examined at the last observed value (LOV).

FIG. 7 shows the total number of new T1 Gd-enhancing lesions when compared annually.

FIG. 8 shows the total number of new T1 Gd-enhancing lesions in the ITT cohort when compared annually.

FIG. 9 shows quantification of the NAA/CR ratio, as measured by MRS, from baseline over 2 years.

### DETAILED DESCRIPTION OF THE INVENTION

This invention provides a method for reducing the frequency of relapses in a patient who experienced a single clinical attack consistent with multiple sclerosis and who has at least one lesion consistent with multiple sclerosis prior to development of clinically definite multiple sclerosis (CDMS), the method consisting essentially of periodically administering to the patient a pharmaceutical composition comprising a pharmaceutically acceptable carrier and an amount of glatiramer acetate therapeutically effective to increase the time to relapse in the patient.

In an embodiment of the method, the time to relapse is delayed by 50%, or by 50% to 115%, or by 60% to 115%, or by 70% to 115%, or by 80% to 115%, or by 90% to 115%, or by 100% to 115%, or by 115%.

In another embodiment of the method, the single clinical attack includes a clinical episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

In another embodiment of the method, the at least one lesion is detectable by an MRI scan and is associated with brain tissue inflammation, myelin sheath damage or axonal damage.

In another embodiment of the method, the lesion is a demyelinating white matter lesion visible on brain MRI.

In another embodiment of the method, the white matter lesions are at least 3 mm in diameter.

In another embodiment of the method, the periodic administration is once-a-day.

In another embodiment of the method, the therapeutically effective amount of glatiramer acetate is 20 mg.

In another embodiment of the method, the administration is subcutaneous.

In another embodiment of the method, the amount of glatiramer acetate is subcutaneously administered via a prefilled syringe.

This invention also provides a method for delaying onset of clinically definite multiple sclerosis in a patient presenting a first clinical event consistent with multiple sclerosis and at least one lesion consistent with multiple sclerosis comprising periodically administering to the patient as monotherapy a pharmaceutical composition comprising a pharmaceutically acceptable carrier and an amount of glatiramer acetate therapeutically effective to delay onset of clinically definite multiple sclerosis.

Additional embodiments of the method are described herein throughout the application.

This invention further provides a method of treating a patient who has experienced a first clinical episode and has MRI features consistent with multiple sclerosis (MS) consisting essentially of subcutaneously administering once a day to the patient prior to conversion to clinically definite multiple sclerosis (CDMS) a pharmaceutical composition comprising mannitol and 20 mg of glatiramer acetate.

This invention provides a method for delaying the onset of clinically definite multiple sclerosis in a patient at risk of developing clinically definite multiple sclerosis, the method comprising periodically administering a pharmaceutical composition comprising a therapeutically effective amount of glatiramer acetate to the patient, thereby delaying onset of clinically definite multiple sclerosis in the patient.

This invention also provides a method for reducing progression of magnetic resonance imaging (MRI)-monitored disease activity in a patient at risk for developing clinically definite multiple sclerosis, the method comprising periodically administering a pharmaceutical composition comprising a therapeutically effective amount of glatiramer acetate to the patient thereby reducing progression of MRI-monitored disease activity in the patient.

This invention further provides a method for reducing the progression of symptoms of Multiple Sclerosis in a patient, the method comprising periodically administering a pharmaceutical composition comprising a therapeutically effective amount of glatiramer acetate to the patient prior to development of clinically definite multiple sclerosis in the patient, thereby reducing the progression of symptoms of MS in the patient.

In an embodiment of the methods onset is delayed by 50% to 115%, or by 604 to 115%, or by 70% to 115%, or by 80% to 115%, or by 90% to 115%, or by 100% to 115%, or 115%.

In another embodiment of the methods, prior to administration, the patient has not experienced a single monofocal or multifocal neurological clinical episode compatible with multiple sclerosis.

In an embodiment of the methods disclosed, prior to administration, the patient has experienced a single clinical attack suggestive of multiple sclerosis.

This invention additionally provides a method for reducing the frequency of relapse in a patient who experienced a single clinical attack suggestive of multiple sclerosis and who has at least one lesion suggestive of multiple sclerosis comprising periodically administering to the patient a pharmaceutical composition comprising an amount of glatiramer acetate therapeutically effective to increase the time to relapse in the patient.

In an embodiment of the methods the time to relapse is increased by 50% to 115%, or by 60% to 115%, or by 70% to 115%, or by 80% to 115%, or by 90% to 115%, or by 100% to 115%, or 115%.

In another embodiment of the methods the single clinical attack includes a clinical episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

This invention also provides a method for delaying progression to clinically definite multiple sclerosis in a patient presenting a first clinical event suggestive of multiple sclerosis and at least one lesion of multiple sclerosis comprising periodically administering to the patient a pharmaceutical composition comprising an amount of glatiramer acetate therapeutically effective to delay progression to clinically definite multiple sclerosis.

In another embodiment of the methods, prior to administration, the patient has at least 1 cerebral lesion detectable by an MRI scan and suggestive of multiple sclerosis.

In another embodiment of the methods the lesion is associated with brain tissue inflammation, myelin sheath damage or axonal damage.

In another embodiment of the methods the lesion is a demyelinating white matter lesion visible on brain MRI.

In another embodiment of the methods the white matter lesions are at least 3 mm in diameter.

In another embodiment of the methods, prior to administration, the patient has no cerebral lesion detectable by a MRI scan.

In another embodiment of the methods the periodic administration is once-a-day.

In another embodiment of the methods the administration is subcutaneous.

In another embodiment of the methods the therapeutically effective amount of glatiramer acetate is 20 mg.

In another embodiment of the methods the therapeutical effective amount of glatiramer acetate is 40 mg.

In another embodiment, the methods further comprise administration of a corticosteroid.

In another embodiment, the methods further comprise administration of a corticosteroid intravenously.

In another embodiment of the methods, progression of symptoms is assessed by multiple sclerosis related disability in the patient as measured by Kurtzke Expanded Disability Status Scale (EDSS) Score, is assessed by relapse rate in the patient, or is assessed by the progression of MRI-monitored disease activity in the patient.

In another embodiment of the methods the MRI-monitored disease activity is the mean cumulative number of T1 Gd-enhancing lesions in the brain of the patient.

In another embodiment of the methods MRI-monitored disease activity is the mean volume of T1 Gd-enhancing lesions in the brain of the patient.

In another embodiment of the methods the MRI-monitored disease activity is the mean cumulative number of T1 hypointense lesions in the brain of the patient.

In another embodiment of the methods MRI-monitored disease activity is the mean volume of hypointense lesions in enhanced T1 weighted images.

In another embodiment of the methods the MRI-monitored disease activity is the mean number of new T2 lesions in the brain of the patient.

In another embodiment of the methods the MRI-monitored disease activity is the mean T2 lesion volume in the brain of the patient.

In another embodiment of the methods the MRI-monitored disease activity is the rate of brain atrophy measured according to the SIENA technique in the patient.

In another embodiment of the methods the glatiramer acetate is administered as monotherapy.

In another embodiment of the methods axonal injury is reduced in the subject.

In another embodiment of the methods the ratio of NAA/CR, as measured in the subject by MRS, increases over time.

In another embodiment of the methods the ratio of NAA/CR, as measured in the subject by MRS, increases to 0.13 with respect to a baseline ratio measured in said subject.

In another embodiment of the methods the frequency of confirmed relapses is reduced over a period of 2-3 years.

In another embodiment of the methods the progression of disease disability is reduced over a period of 2-3 years.

In another embodiment of the methods the rate of accumulating new T2-weighted lesions is reduced by at least 50%, as compared to a subject not treated with glatiramer acetate. In an additional embodiment the rate of accumulating new T2-weighted lesions is reduced by 50-90%, as compared to a subject not treated with glatiramer acetate. In a further embodiment the rate of accumulating new T2-weighted lesions is reduced by 50-60%, as compared to a subject not treated with glatiramer acetate. In yet another embodiment the rate of accumulating new T2-weighted lesions is reduced by 53%, as compared to a subject not treated with glatiramer acetate.

In another embodiment of the methods the number of new T2 lesions occurring annually is reduced, as compared to a subject not treated with glatiramer acetate.

In another embodiment of the methods the number of new T1 Gd-enhancing lesions is reduced by at least 50%, as compared to a subject not treated with glatiramer acetate. In an additional embodiment the number of new T1 Gd-enhancing lesions is reduced by 50-90%, as compared to a subject not treated with glatiramer acetate. In a further embodiment the number of new T1 Gd-enhancing lesions is reduced by 50-65%, as compared to a subject not treated with glatiramer acetate. In yet another embodiment the number of new T1 Gd-enhancing lesions is reduced by 61%, as compared to a subject not treated with glatiramer acetate.

In another embodiment of the methods the subject is female and the risk to conversion to CDMS is reduced by at least 40%, as compared to a subject not treated with glatiramer acetate. In an additional embodiment the subject is female and the risk to conversion to CDMS is reduced by 40-60%, as compared to a subject not treated with glatiramer acetate. In a further embodiment the subject is female and the risk to conversion to CDMS is reduced by 45-55%, as compared to a subject not treated with glatiramer acetate. In yet another embodiment the subject is female and the risk to conversion to CDMS is reduced by 48%, as compared to a subject not treated with glatiramer acetate.

In another embodiment of the methods the subject is male and the risk to conversion to CDMS is reduced by at least 35%, as compared to a subject not treated with glatiramer acetate. In an additional embodiment the subject is male and the risk to conversion to CDMS is reduced by 35-60%, as compared to a subject not treated with glatiramer acetate. In a further embodiment the subject is male and the risk to conversion to CDMS is reduced by 40-50%, as compared to a subject not treated with glatiramer acetate. In yet another embodiment the subject is male and the risk to conversion to CDMS is reduced by 43%, as compared to a subject not treated with glatiramer acetate.

In another embodiment of the methods the subject is less than 30 years old and the risk to conversion to CDMS is reduced by at least 40%, as compared to a subject not treated with glatiramer acetate. In an additional embodiment the subject is less than 30 years old and the risk to conversion to CDMS is reduced by 40-60%, as compared to a subject not treated with glatiramer acetate. In a further embodiment the subject is less than 30 years old and the risk to conversion to CDMS is reduced by 50-60%, as compared to a subject not treated with glatiramer acetate. In yet another embodiment the subject is less than 30 years old and the risk to conversion to CDMS is reduced by 53%, as compared to a subject not treated with glatiramer acetate.

In another embodiment of the methods the subject is greater than 30 years old and the risk to conversion to CDMS is reduced by at least 25%, as compared to a subject not treated with glatiramer acetate. In an additional embodiment the subject is greater than 30 years old and the risk to conversion to CDMS is reduced by 25-45%, as compared to a subject not treated with glatiramer acetate. In a further embodiment the subject is greater than 30 years old and the risk to conversion to CDMS is reduced by 30-45%, as compared to a subject not treated with glatiramer acetate. In yet another embodiment the subject is greater than 30 years old and the risk to conversion to CDMS is reduced by 37%, as compared to a subject not treated with glatiramer acetate.

In another embodiment of the methods the subject was treated with corticosteroid for the initial attack and the risk of conversion to CDMS is reduced by at least 30%, as compared to a subject not treated with glatiramer acetate. In an additional embodiment the subject was treated with corticosteroid for the initial attack and the risk of conversion to CDMS is reduced by 30-50%, as compared to a subject not treated with glatiramer acetate. In a further embodiment the subject was treated with corticosteroid for the initial attack and the risk of conversion to CDMS is reduced by 0%, as compared to a subject not treated with glatiramer acetate. In yet another embodiment the subject was treated with corticosteroid for the initial attack and the risk of conversion to CDMS is reduced by 39%, as compared to a subject not treated with glatiramer acetate.

In another embodiment of the methods the subject was not treated with corticosteroid for the initial attack and the risk of conversion to CDMS is reduced by at least 45%, as compared to a subject not treated with glatiramer acetate. In an additional embodiment the subject was not treated with corticosteroid for the initial attack and the risk of conversion to CDMS is reduced by 45-85%, as compared to a subject not treated with glatiramer acetate. In a further embodiment the subject was not treated with corticosteroid for the initial attack and the risk of conversion to CDMS is reduced by 50-60%, as compared to a subject not treated with glatiramer acetate. In yet another embodiment the subject was not treated with corticosteroid for the initial attack and the risk of conversion to CDMS is reduced by 54%, as compared to a subject not treated with glatiramer acetate.

In another embodiment of the methods the subject presents with unifocal optic manifestation and the risk of conversion to CDMS is reduced by at least 55%, as compared to a subject not treated with glatiramer acetate. In an additional embodiment the subject presents with unifocal optic manifestation and the risk of conversion to CDMS is reduced by 55-85%, as compared to a subject not treated with glatiramer acetate. In a further embodiment the subject presents with unifocal optic manifestation and the risk of conversion to CDMS is reduced by 55-75%, as compared to a subject not treated with glatiramer acetate. In yet another embodiment the subject presents with unifocal optic manifestation and the risk of conversion to CDMS is reduced by 66%, as compared to a subject not treated with glatiramer acetate.

In another embodiment of the methods the subject presents with T1 Gd-enhanced lesions and the risk of conversion to CDMS is reduced by at least 60%, as compared to a subject not treated with glatiramer acetate. In an additional embodiment the subject presents with T1 Gd-enhanced lesions and the risk of conversion to CDMS is reduced by 60-90%, as compared to a subject not treated with glatiramer acetate. In a further embodiment the subject presents with T1 Gd-enhanced lesions and the risk of conversion to CONS is reduced by 65-80%, as compared to a subject not treated with glatiramer acetate. In yet another embodiment the subject presents with T1 Gd-enhanced lesions and the risk of conversion to CDMS is reduced by 71%, as compared to a subject not treated with glatiramer acetate.

In another embodiment of the methods the subject presents with 9 or more T2 lesions and the risk of conversion to CDMS is reduced by at least 50%, as compared to a subject not treated with glatiramer acetate. In an additional embodiment the subject presents with 9 or more T2 lesions and the risk of conversion to CDMS reduced by 50-90%, as compared to a subject not treated with glatiramer acetate. In a further embodiment the subject presents with 9 or more T2 lesions and the risk of conversion to CDMS is reduced by 50-60%, as compared to a subject not treated with glatiramer acetate. In yet another embodiment the subject presents with 9 or more T2 lesions and the risk of conversion to CDMS is reduced by 58%, as compared to a subject not treated with glatiramer acetate.

In another embodiment of the methods the subject does not present with T1 Gd-enhanced lesions and the risk of conversion to CDMS is reduced by at least 35%, as compared to a subject not treated with glatiramer acetate. In an additional embodiment the subject does not present with T1 Gd-enhanced lesions and the risk of conversion to CDMS is reduced by 35-65%, as compared to a subject not treated with glatiramer acetate. In a further embodiment the subject does not present with T1 Gd-enhanced lesions and the risk of conversion to CDMS is reduced by 40-50%, as compared to a subject not treated with glatiramer acetate. In yet another embodiment the subject does not present with T1 Gd-enhanced lesions and the risk of conversion to CDMS is reduced by 44%, as compared to a subject not treated with glatiramer acetate.

In another embodiment of the methods the subject presents with less than 9 T2 lesions and the risk of conversion to CDMS is reduced by at least 55%, as compared to a subject not treated with glatiramer acetate. In an additional embodiment the subject presents with less than 9 T2 lesions and the risk of conversion to CDMS is reduced by 55-85%, as compared to a subject not treated with glatiramer acetate. In a further embodiment the subject presents with less than 9 T2 lesions and the risk of conversion to CDMS is reduced by 65-75%, as compared to a subject not treated with glatiramer acetate. In yet another embodiment the subject presents with less than 9 T2 lesions and the risk of conversion to CDMS is reduced by 67%, as compared to a subject not treated with glatiramer acetate.

This invention further provides a use of glatiramer acetate in the manufacture of a medicament for delaying the onset of clinically definite multiple sclerosis, for reducing progression of magnetic resonance imaging (MRI)-monitored disease activity, or reducing progression of symptoms of multiple sclerosis in a patient at risk for developing clinically definite multiple sclerosis.

This invention also provides a use of glatiramer acetate in the manufacture of a medicament for the treatment of a patient who experienced a single demyelinating event and an active inflammatory process, which are indicative of the patient being at high risk of developing clinically definite multiple sclerosis.

This invention further provides glatiramer acetate for use in treating of a patient who experienced a first clinical event suggestive of multiple sclerosis and is at risk of developing clinically definitive multiple sclerosis.

This invention yet further provides use of glatiramer acetate in the manufacture of a medicament for the treatment of a patient who experienced a first clinical event suggestive of multiple sclerosis and is at risk of developing clinically definite multiple sclerosis.

All combinations of the various elements described herein are within the scope of the invention.

### DEFINITIONS

As used herein, a patient at risk of developing MS (i.e. clinically definite MS) is a patient presenting any of the known risk factors for MS. The known risk factors for MS include any one of a clinically isolated syndrome (CIS), a single attack suggestive of MS without a lesion, the presence of a lesion (in any of the CNS, PNS, or myelin sheath) without a clinical attack, environmental factors (geographical location, climate, diet, toxins, sunlight) (16, 17, 18), genetics (variation of genes encoding HLA-DRB1, IL7R-alpha and IL2R-alpha) (19, 20), and immunological components (viral infection such as by Epstein-Barr virus, high avidity CD4 T cells, CD8+ T cells, anti-NE-L, anti-CSF114(Glc)) (21, 22, 23). As used herein, clinically isolated syndrome (CIS) refers to 1) a single clinical attack (used interchangeably herein with “first clinical event” and “first demyelinating event”) suggestive of MS, which, for example, presents as an episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning, and 2) at least one lesion suggestive of MS. In a specific example, CIS diagnosis would be based on a single clinical attack and at least 2 lesions suggestive of MS measuring 6 mm or more in diameter.

As used herein, the criteria as defined by Poser et al. (1) used to determine if a subject meets the condition consistent with clinically definite MS (CDMS) are:
Two attacks and clinical evidence of two separate lesions or
Two attacks; clinical evidence of one lesion and paraclinical evidence of another separate lesion.

An attack (also referred to as an exacerbation, flare, or relapse,) is defined clinically as the sudden appearance or worsening of a symptom or symptoms of neurological dysfunction, with or without objective confirmation.

Clinical evidence of a lesion is defined as signs of neurological dysfunction demonstrable by neurological examination. An abnormal sign constitutes clinical evidence even if no longer present, but was recorded in the past by a competent examiner.

Paraclinical evidence of a lesion is defined as the demonstration by means of various tests and procedures of the existence of a lesion of the CNS that has not produced clinical signs but that may or may not have caused symptoms in the past. Such evidence may be derived from the hot-bath test, evoked response studies, neuroimaging, and expert neurological assessment. These tests are considered to be extensions of the neurological examination and not laboratory procedures. (The term ‘paraclinical’ meaning beside, alongside of, or associated in a subsidiary or accessory capacity (Webster's Unabridged Dictionary), was chosen instead of ‘subclinical’.) (13)

As used herein, the SIENA (Structural Image Evaluation of Normalized Atrophy) method (14) is used for measuring brain atrophy in patients. Brain atrophy constantly occurs and progressively increases in MS patients due to axonal damage, Demyelination and inflammation. In the SIENA longitudinal method, the external surface of the skull is used as an invariant constraint on serial images, which is usually clearly visible on T1-weighted images. The brain is segmented from non-brain, using 3D triangulated mesh modeling to the brain surface, a procedure that balances local and global constraints and uses a local threshold and smoothness factor to reliably detect the brain surface. Once the brain surface is found on one scan, the program then finds surface point positions to sub-voxel accuracy (between scans at two different time points) using correlation of normal vectors. This is then converted into percentage brain volume change (PBVC). The precision and accuracy of PBVC is around 0.2%; better precision is achieved with thicker slices, perhaps because sequence acquisition time is less, thereby reducing motion artifacts.

As used herein, the term Gd-enhancing lesions, refers to lesions that result from a breakdown of the blood brain barrier, which appear in contrast studies using gadolinium contrast agents. Gadolinium enhancement provides information as to the age of a lesion, as Gd-enhancing lesions typically occur within a six week period of lesion formation.

As used herein, the term T1-weighted MRI image, refers to an MR-image that emphasizes T1 contrast by which lesions may be visualized. Abnormal areas in a T1-MRI weighted image are “hypointense” and appear as dark spots. These spots are generally older lesions.

As used herein, the term T2-weighted MRI image, refers to an MR-image that emphasizes T2 contrast by which lesions may be visualized. T2 lesions represent new inflammatory activity.

As used herein, neurological dysfunction refers to any one of the following indications (14): blurring of vision, diplopia, optic neuritis, involuntary rapid eve movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems (including urgency, frequency, incomplete emptying and incontinence), bowel problems (including constipation and loss of bowel control), impotence, diminished sexual arousal, loss of sensation, sensitivity to heat, loss of short term memory, loss of concentration, or loss of judgment or reasoning.

This invention is illustrated in the Examples section which follows. This section is set forth to aid in an understanding of the invention but is not intended to, and should not be construed to limit in any way the invention as set forth in the claims which follow thereafter.

### EXPERIMENTAL DETAILS

### Example 1

### Evaluating Effect of Glatiramer Acetate (GA) Treatment in Patients Presenting a Clinically Isolated Syndrome (CIS) on the Time to Conversion to CDMS

A clinical trial was undertaken to assess the effect of treatment with GA compared to placebo on the time to conversion to CDMS, as determined by Poser (the occurrence of the second clinical attack) during the double-blind phase.

### Methods

481 subjects between the ages of 18 and 45 years, with a single well-defined unifocal neurological event suggestive of MS, and exhibiting at least 2 cerebral lesions suspicious of MS on the screening MRI measuring 6 mm or more in diameter, are included and randomized in equal numbers to receive 20 mg GA or placebo. Subjects receive their first dose of study medication at the baseline visit. 20 mg GA formulation is injected once daily by subcutaneous route via pre-filled syringe manufactured by Teva Pharmaceutical Industries Ltd., Israel. Subjects are evaluated at study centers at baseline, at months 1, 3, and every 3 months thereafter.

The duration of the double-blind phase is 36 months (3 years) or until subject's conversion to CDMS, whichever comes first. Conversion to CDMS is counted when the subject's symptoms are accompanied by observed objective neurological changes, consistent with:
a) an increase of at least 0.5 in the EDSS score or one grade in the score of two or more of the seven Functional Systems (FS); or
b) two grades in the score of one of FS as compared to the previous evaluation.

The subject must not be undergoing any acute metabolic changes such as fever or other medical abnormality. A change in bowel/bladder function or in cognitive function must not be entirely responsible for the changes in EDSS or FS scores.

### Results

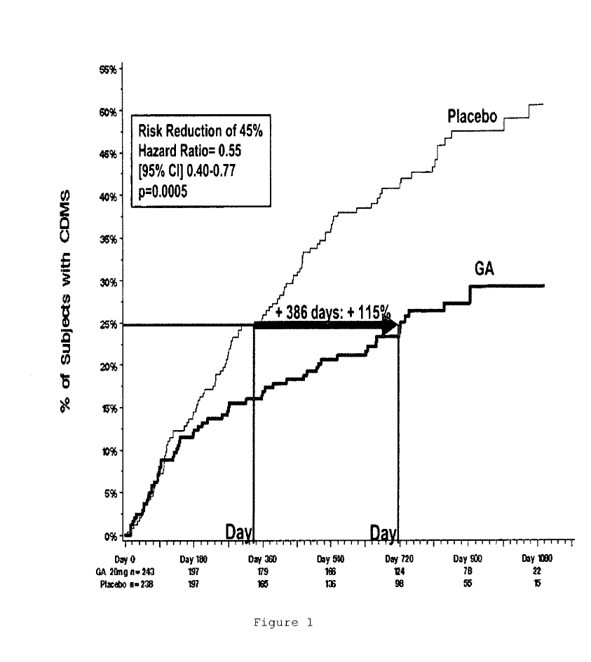

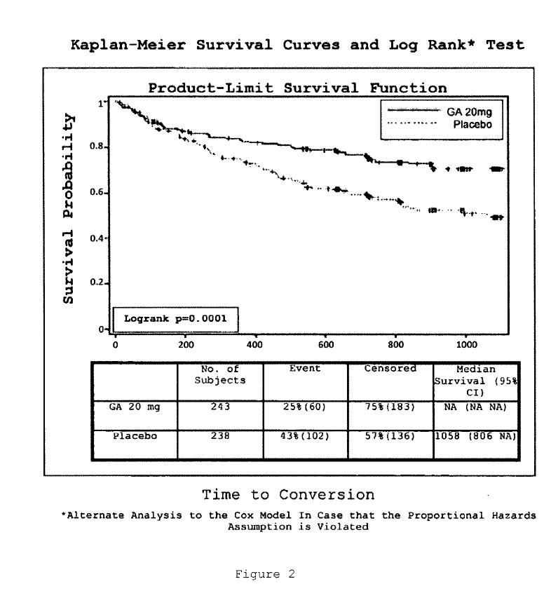

During the study period, GA treatment delayed the conversion to clinically definite MS. Specifically, the study involving a total of 481 subjects randomized to the two study arms demonstrated prolongation of the quartile time to CDMS by 115%, from 336 days for placebo to 722 days for GA treatment. Glatiramer acetate reduced the risk in developing clinically definite MS (CDMS) by 44% (Hazard Ratio 0.56). Detailed experimental data is present in tables 1 and 2 and in FIGS. 1 and 2. FIG. 2 shows the Kaplan-Meier survival curves and log rank test by an alternative analysis to the Cox Model in case that the proportional hazards assumption is violated.

TABLE 1
Analysis of Primary Efficacy Endpoint; Cox
Model Summary Results of Time to CDMS

| | | 95% Lower | 95% Upper | |
|---|---|---|---|---|
| | | Confidence | Confidence | |
| | | Limit for | Limit for | |
| GA 20 mg vs Placebo | Hazard | Hazard | Hazard | Pr > |
| Data Analysis Set | Ratio | Ratio | Ratio | ChiSq |
| ITT (481 Patients) | 0.555 | 0.396 | 0.770 | 0.0005 |
| Completers (423 | 0.581 | 0.414 | 0.815 | 0.0017 |
| Patients) | | | | |
| ITT + Available | 0.556 | 0.399 | 0.774 | 0.0005 |
| Follow-UP (481 Patients) | | | | |

P-value of Cox Proportional Hazards Assumption Test = 0.33 → Proportional Hazards Assumption is NOT Violated

TABLE 2
Kaplan-Meier Product Limit Survival Time Percentiles
Estimate (Days) ITT Data Analysis set

| | Kaplan Meier Survival Time | | |
|---|---|---|---|
| | Estimate (Days) | | |
| Percentile Estimate and | GA 20 mg | Placebo | |
| 95% CI | (N = 243) | (N = 238) | Difference |
| 29% Percentile | 903 | 416 | 487 (46%) |
| Lower Limit of 95% CI for | 658 | 280 | |
| 29% Percentile | | | |
| Upper Limit of 95% CI for | | 526 | |
| 29% Percentile | | | |
| 25% Percentile | 722 | 336 | 386 (47%) |
| Lower Limit of 95% CI for | 505 | 260 | |
| 25% Percentile | | | |
| Upper Limit of 95% CI for | | 456 | |
| 25% Percentile | | | |
| 20% Percentile | 505 | 260 | 245 (51%) |
| Lower Limit of 95% CI for | 271 | 186 | |
| 20% Percentile | | | |
| Upper Limit of 95% CI for | 733 | 359 | |
| 20% Percentile | | | |

### CONCLUSION

Treatment with GA in persons presenting a CIS suggestive of MS significantly delayed the development of clinically definite MS.

### Example 2

### Evaluating Effect of Glatiramer Acetate (GA) Treatment Patients Presenting a Clinically Isolated Syndrome (CIS) on Clinical and MRI Parameters

A clinical trial was undertaken to assess, within the time frame of the up to 3-years placebo-controlled study period, the effect of GA on clinical and MRI parameters.

### Methods

481 subjects between the ages of 18 and 45 years, with a single well-defined unifocal neurological event highly suggestive of MS, and exhibiting at least 2 cerebral lesions highly suspicious of MS on the screening MRI measuring 6 mm or more in diameter, are included and randomized in equal numbers to receive 20 mg GA or placebo. Subjects received their first dose of study medication at the baseline visit. 20 mg GA formulation was injected once daily by subcutaneous route via pre-filled syringe manufactured by Teva Pharmaceutical Industries Ltd., Israel. The duration of the double-blind phase is 36 months (3 years) or until subject's conversion to CDMS, whichever comes first.

The effect of GA treatment relative to placebo during the double-blind phase on clinical and MRI parameters is assessed as follows: proportion of patients who convert to CDMS; the total number of new T2 lesions observed at the last scan taken during the placebo-controlled phase; total number of new T2 lesions annually; total number of new T2 lesions annually in the ITT cohort; the total number of new T1 Gd-enhancing lesions observed at the last scan taken during the placebo-controlled phase; total number of new T1 Gd-enhancing lesions annually; total number of new T1 Gd-enhancing lesions annually in the ITT cohort; brain atrophy as defined by the change from baseline to the last scan taken during the double-blind phase in brain volume measured according to the SIENA technique (14).

### Results

During the study period GA treatment reduced the rate of development of clinically definite MS, reduces accumulation of new MRI-detected lesions in the brain, and reduces the level of brain atrophy. Specifically, based on the Kaplan-Meier estimates, the probability of development of CDMS over 3 years is reduced by treatment from 65% in the placebo group to 36.4% in the GA group. At the end of the two-year study, 25 percent of patients in the treatment group developed CMS compared to 43 percent of the placebo group. Moreover, the number of new MRI detected lesions is significantly lower in the GA treatment group as follows:

### Total Number of New T2 Lesions (LOV)

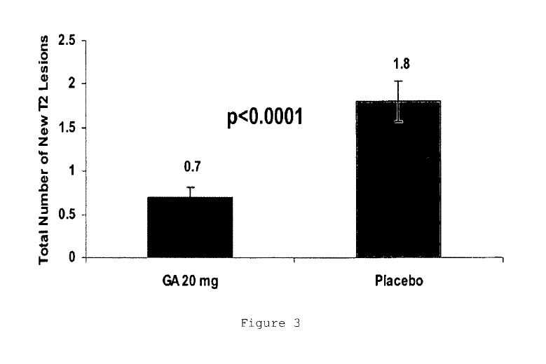

Patients receiving glatiramer acetate experienced a significant reduction in the cumulative number of new T2-weighted lesions when examined at the last observed value (LOV) of the placebo controlled phase. The results reflect a treatment effect of 58% in decreasing the rate of new T2 lesions with glatiramer acetate treatment (0.7 in patients treated with 20 mg glatiramer acetate in comparison to 1.8 in the placebo group; see FIG. 3 and Table 3).

TABLE 3

Number of New T2 Lesions at LOV

| | GA 20 mg (N = 243, | Placebo (N = 238, |
|---|---|---|
| GA/9010 | Subject- | Subject- |
| (PreCISe) | Years = 431.4) | Years = 381.5) |
| N | 220 | 221 |
| Mean | 0.7 | 1.8 |
| SD | 1.7 | 3.6 |
| Min | 0 | 0 |
| Median | 0 | 0 |
| Max | 15 | 19 |

### Total Number of New T2 Lesions Compared Annually

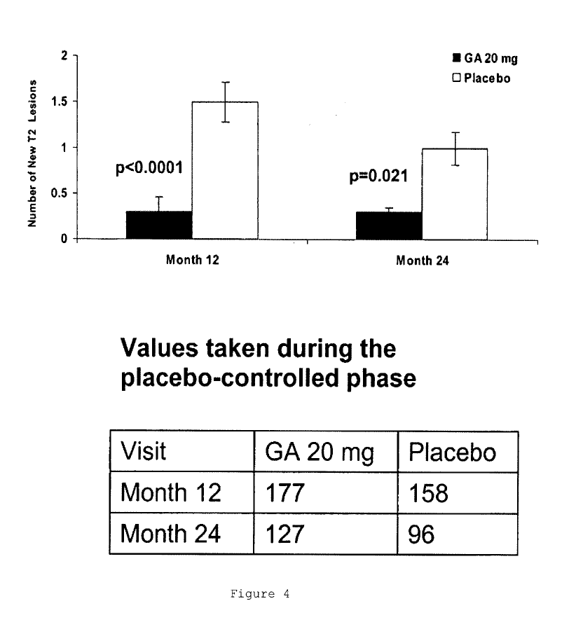

Annual comparison of new T2 lesions shows that patients benefited from a 6-fold reduction in comparison to the placebo group when examined at 12 months. At 24 months patients continued to have a reduced number of new T2 lesions (4-fold) in comparison to the placebo group (see FIG. 4).

### Total Number of New T2 Lesions Compared Annually (ITT Cohort)

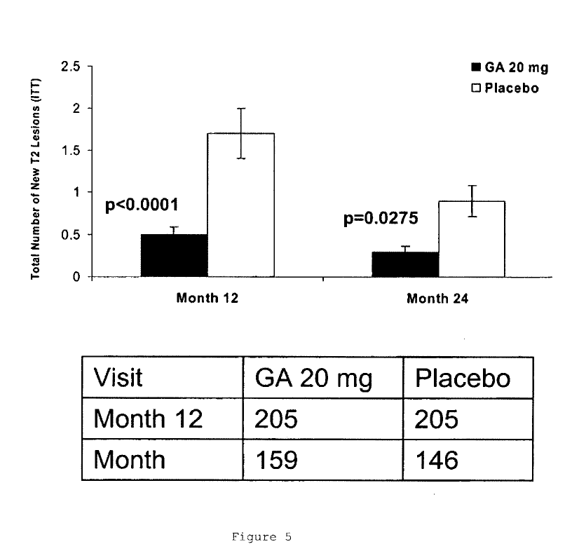

Annual comparison of new T2 lesions within the ITT cohort shows that patients benefited from over a 3-fold reduction in comparison to the placebo group when examined at 12 months. At 24 months patients continued to have a reduced number of new T2 lesions (approximately 4-fold) in comparison to the placebo group (see FIG. 5).

### Total Number of New T1 Gd-Enhancing Lesions (LOV)

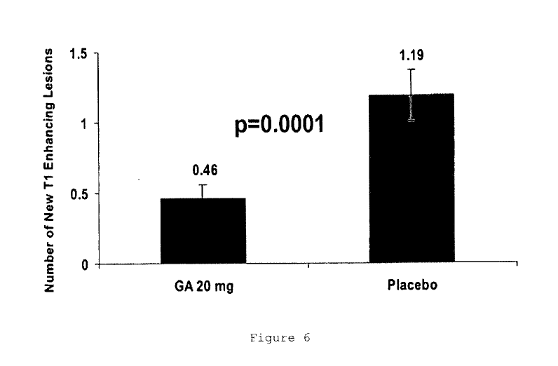

Glatiramer acetate was also effective in reducing the cumulative number of new T1 Gd-enhancing lesions at the last observed value (LOV) by 619 when compared to the placebo group (0.46 in patients treated with glatiramer acetate and 1.19 in the placebo group; see FIG. 6).

### Total Number of New T1 Gd-Enhancing Lesions Compared Annually

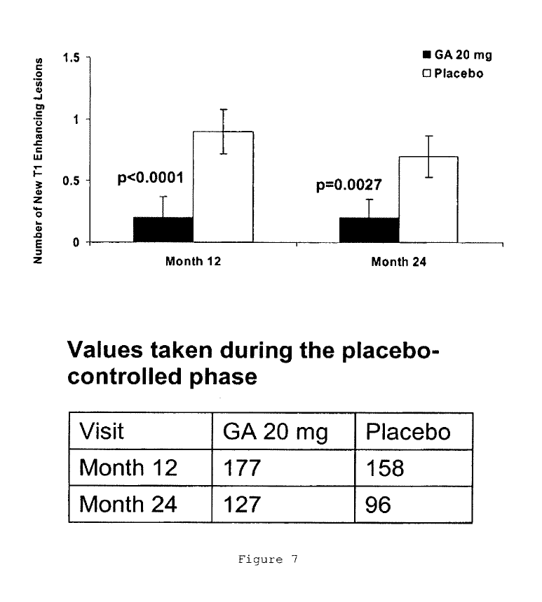

Annual comparison of new T1 Gd-enhancing lesions shows that patients benefited from over a 4.8-fold reduction in comparison to the placebo group when examined at 12 months. At 24 months patients continued to have a reduced number of new T2 lesions (approximately 3.8-fold) in comparison to the placebo group (see FIG. 7).

### Total Number of New T1 Gd-Enhancing Compared Annually (ITT Cohort)

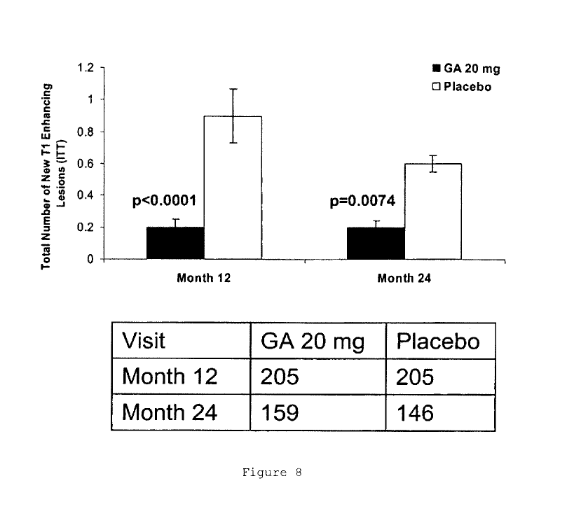

Annual comparison of new T1 Gd-enhancing lesions within the ITT cohort shows that patients benefited from over a 4.5-fold reduction in comparison to the placebo group when examined at 12 months. At 24 months patients continued to have a reduced number of new 12 lesions (approximately 3-fold) in comparison to the placebo group (see FIG. 8).

### Conclusions

Over a 3 year period, treatment with GA in persons presenting a CIS suggestive of multiple sclerosis significantly reduced the rate of development of clinically definite MS, reduces occurrence of new MRI-detected lesions in the brain, reduces accumulation of lesion area in the brain and reduces brain atrophy relative to persons taking placebo. These results show that GA treatment in persons at high risk for developing MS is an effective method of reducing the occurrence of clinically definite MS and of preventing irreversible brain damage in these persons.

### Example 3

### Evaluating Effect of Glatiramer Acetate (GA) Treatment in Patients Representing Different Demographics and Subgroups

Subgroup analyses related to the primary efficacy variable were performed with respect to demographics and CIS characteristics at initial attack onset (gender, age, and type of unifocal manifestation and steroid treatment for the initial attack), and MRI findings (disease dissemination/activity) at study baseline.

Four years after the study was initiated and a few months before the Statistical Analysis Plan (SAP) for the Interim Analysis (IA) was finalized, the European Medicines Agency (EMEA) revised guideline for conducting studies in MS came into effect (June, 2007). The revised version refers to studies in a CIS population as follows: “In CIS, the delay of the occurrence of a second clinical attack, although relevant from a mechanistic perspective, is of limited clinical relevance. It is needed to demonstrate efficacy by means of a meaningful and sustained relapse rate over 2-3 year time and it is recommended to assess the decrease of the accumulation of disability . . . . In patients with CIS the relapse rate and the percentage of patients with no further relapses are preferred efficacy variables instead of the second clinical event. As in other MS forms, accumulation of disability is considered a relevant efficacy parameter that should be evaluated”.

In view of the above, post-hoc analyses were performed for the following endpoints:
Number of confirmed relapses
Progression of disease disability

No correction for multiplicity was done for any of the following post-hoc analyses.
1. Subgroup analysis of the primary endpoint for: gender, age, type of unifocal manifestation and corticosteroid use for the initial attack employed the Cox proportional hazards model, as for the principal analysis. Subgroup analyses of proportion of subjects converted to CDMS according to MRI activity at baseline was analyzed using Logistic Regression, as in the fourth secondary endpoint.
2. Number of relapses: analysis of number of relapses during the placebocontrolled phase, during the entire study and on a yearly basis was performed using the Poisson regression.
3. Time to confirmed Expanded Disability Status Scale (EDSS) progression: Progression of disability was defined as worsening of at least 1 point in EDSS sustained over 2 consecutive measurements which are at least 6 months apart. Analysis of time to confirmed EDSS progression was performed employing the Cox proportional hazards model.

Due to the trial design, where all placebo subjects switched to active treatment upon conversion to CDMS (Poser) or after 3 years in study, endpoints that depend on exposure duration to the drug are potentially biased. Therefore this endpoint was calculated and analyzed only for the entire study period data (placebo-controlled and open-label phases combined) available by the cut-off date of the IA.

Baseline demographic and disease characteristics were comparable between the 2 groups. The study consisted of 65.4% females and 34.6% males on Copaxone® compared to 68.5% females and 31.5% males on placebo. The mean (SD) age was 31.5(6.9) years for the Copaxone® group and 30.8(7.0) for placebo. The treatment groups were comparable in their CIS characteristics: time since first symptom, distribution of the outcome of first symptom and distribution of type of unifocal manifestation at initial attack. For about a third of the subjects in each group, the unifocal manifestation was of cerebral origin, for a third it was of optic origin, for 19% it was of spinal origin, and for ˜12% it was undeterminable whether it was of spinal or cerebral origin. MRI measures at baseline were comparable for the two groups (see also Table 4). EDSS scores at baseline (Table 4) were similar for both groups [median 1.00; range of 0.0-5.0)]

TABLE 4
Distribution of Subjects by Subgroups

| | | | |
|---|---|---|---|
| | GA 20 mg | Placebo | ALL |
| | (N = 243) | (N = 238) | (N = 481) |
| | N (%) | N (%) | N (%) |
| GA/9010 (PreCISe) | Subjects | Subjects | Subjects |

Demographics and CIS Characteristics at Onset

| | | | | |
|---|---|---|---|---|
| Gender | Female | 159 (65%) | 163 (69%) | 322 (67%) |
| | Male | 84 (35%) | 75 (32%) | 159 (33%) |
| Age | <30 years | 109 (45%) | 118 (50%) | 227 (47%) |
| | >=30 years | 134 (55%) | 120 (50%) | 254 (53%) |
| Corticosteroids | Yes | 149 (61%) | 159 (67%) | 308 (64%) |
| Use for | No | 94 (39%) | 79 (33%) | 173 (36%) |
| Initial Attack | | | | |
| Type of | Cerebral | 83 (34%) | 84 (35%) | 167 (35%) |
| Unifocal | Cerebral or | 30 (12%) | 26 (11%) | 56 (12%) |
| Manifestation | Spinal | | | |
| | Optic | 82 (34%) | 86 (36%) | 168 (35%) |
| | Spinal | 48 (20%) | 42 (18%) | 90 (19%) |

MRI findings at Study Baseline

| | | | | |
|---|---|---|---|---|
| # of T1 Gd- | T1 = 0 lesions | 144 (60%) | 126 (53%) | 270 (56%) |
| enhancing | T1 >= 1 lesions | 98 (41%) | 111 (47%) | 209 (44%) |
| Lesions at | | | | |
| Baseline | | | | |
| # of T2 | 2-8 lesions | 37 (15%) | 38 (16%) | 75 (16%) |
| Lesions at | >=9 lesions | 205 (85%) | 199 (84%) | 404 (84%) |
| Baseline | | | | |

### Results

The study population of 481 subjects (Copaxone®: n=243; placebo: n=238) were divided post-hoc into subgroups for analyzing the primary endpoint, the risk in three years for conversion to CDMS. Subgroups were created for demographics and CIS characteristics at onset (gender, age, and type of unifocal presentation and steroid treatment for the initial attack), and MRI findings (disease dissemination/activity) at study baseline. The results are summarized in table 4.

As the subgroup of cerebral or spinal clinical presentation was small, the analysis was performed only for the 3 other subtypes of unifocal manifestation.

Subgroup analyses of the risk for conversion to CDMS in three years according to demographic and disease baseline factors demonstrated significant effects for Copaxone®. in most of the subgroups evaluated (Table 5 and Table 6).

TABLE 5
Time to CDMS in the Placebo-Controlled Phase by Demographics
and CIS Characteristics at Onset: CoxProportional Hazard Model

| | | Copaxone ® | Placebo | | | Risk Reduction |
|---|---|---|---|---|---|---|
| | | (N = 243) | (N = 238) | Hazard Ratio | p- | with Copaxone ® |
| GA/9010 (PreCISe) | | % CDMS | % CDMS | [95% CI] | value | over Placebo |
| Sex | Female | 14% | 29% | 0.52 [0.34, 0.81] | 0.0037 | 48% |
| | Male | 11% | 14% | 0.57 [0.32, 1.02] | 0.0593 | 43% |
| Age (years) | <30 | 10% | 22% | 0.47 [0.27, 0.80] | 0.006 | 53% |
| | >=30 | 15% | 21% | 0.63 [0.40, 1.01] | 0.0531 | 37% |
| Corticosteroids Use | Yes | 16% | 28% | 0.61 [0.40, 0.92] | 0.0191 | 39% |
| for Initial Attack | No | 9% | 15% | 0.46 [0.26, 0.82] | 0.0086 | 54% |
| Type of Unifocal | Cerebral | 10% | 18% | 0.62 [0.36, 1.08] | 0.0923 | 38% |
| Manifestation | Optic | 6% | 12% | 0.34 [0.17, 0.68] | 0.0022 | 66% |
| | Spinal | 7% | 8% | 0.83 [0.38, 1.79] | 0.632 | 17% |

TABLE 6
Proportion of Subjects with CDMS in the Placebo-Controlled Phase by
MRI Activity Subgroups at Study Baseline: Logistic Regression

| | | Copaxone ® | Placebo | | | Risk Reduction |
|---|---|---|---|---|---|---|
| | | (N = 243) | (N = 238) | Odds Ratio | | with Copaxone ® |
| GA/9010 (PreCISe) | | % CDMS | % CDMS | [95% CI] | p-value | over Placebo |
| # of T1 Gd-enhancing | T1 = 0 lesions | 14% | 19% | 0.56 [0.32, 0.98] | 0.0423 | 44% |
| lesions at Baseline | T1 >= 1 | 11% | 25% | 0.29 [0.16, 0.54] | <0.0001 | 71% |
| | lesions | | | | | |
| # of T2 lesions at | 2-8 lesions | 3% | 6% | 0.33 [0.10, 1.05] | 0.0598 | 67% |
| Baseline | >=9 lesions | 22% | 38% | 0.42 [0.27, 0.64] | <0.0001 | 58% |

Using Cox proportional hazard model, as for the principal analysis, a significant risk reduction of 48% was demonstrated for females and 53% for young patients (<30 years); a borderline significant risk reduction of 43% for males and 37% for patients over 30 years was obtained. A significant risk reduction of 39% and 54% was obtained for patients with or without corticosteroid treatment for the initial attack, respectively, and 66% risk reduction was demonstrated for patients presenting with unifocal optic manifestation (Table 5).

The results of the logistic regression comparing Copaxone.RTM. treatment vs. placebo in reference to MRI disease activity at baseline (Table 6) demonstrated significant and pronounced effects of Copaxone® for patients with MRI active disease. A risk reduction of 71% for patients with T1 gadolinium (Gd-) enhancement and 58% for patients with 9 or more T2 lesions were obtained. Copaxone®. was also effective in patients with less MRI active disease at randomization. Patients with no enhancement had a significant risk reduction of 44% and those with less than 9 T2 lesions showed a borderline significant risk reduction of 67%.

### Example 4

### Analysis of Axonal Integrity in Patients with Multiple Sclerosis (MS) and Treated with Glatiramer Acetate by Magnetic Resonance Spectroscopy (MRS)

Magnetic resonance spectroscopy (MRS) provides a non-invasive in-vivo method of quantifying diffuse axonal injury, which is not captured by the conventional lesion-oriented burden of disease metrics. MRS studies have demonstrated loss of axonal integrity in patients with multiple sclerosis (MS), even in the early stages of the disease. The MRS analysis allows investigation as to whether treatment with glatiramer acetate in subjects with clinically isolated syndrome (CIS) suggestive of MS can reduce or delay axonal damage.

Single voxel magnetic resonance spectroscopy (MRS) exams were performed at baseline and once a year subsequently. Scans were quantified locally and sent to the MRS Unit (Montreal) where they were deemed acceptable or in need of repeat (either acquisition or analysis). The MRS endpoint is the change in the ratio of N-acetylaspartate/creatine (NAA/Cr) ratio over time. NAA is seen only in neuronal tissue and is a marker of neuronal integrity; reducing with most types of insults to the brain. Cr is often used as an internal reference because it is relatively stable.

MRS scans were performed after T2-weighted fast-spin-time echo (FSE/TSE) scans and before gadolinium injection. MRS data was obtained from a region of central white matter using a 90-180-180 (PRESS) volume selective sequence to excite a volume of 100 mm×100 mm×20 mm (range 80-100 mm×80-100 mm×20 mm) centered on the body of the corpus callosum using a long echo time (TR 2000, TE 272). The rotation of the acquisition region was the same as for the main image series. The slice region was positioned on the T2-weighted FSE/TSE slice that passes through the superior part of the corpus callosum, one slice above the most superior slice on which the lateral ventricles are visible. The region was centered left-right so that the brain mid-line passes centrally through the region. The region was positioned anterior-posterior so that the anterior corners and posterior corners are equidistant from the skull.

### Results

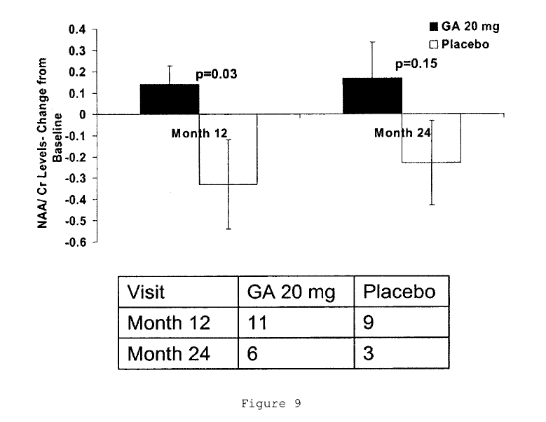

Quantification of the NAA/CR ratio from baseline over time demonstrates the protective and regenerative effects of glatiramer acetate. Treatment with glatiramer acetate reduces axonal damage and helps to preserve neurons in the brain, even at early stages of the disease. Glatiramer acetate treated patients showed a significant increase (approximately 0.15) with respect to the NCAA/Cr ratio at 12 and 24 months, whereas the placebo group should dramatic reductions in NCAA/Cr over time from the baseline value (approximately −0.35 and −0.25 at 12 and 24 months, respectively; see FIG. 9)

### Example 5

### Effect of Glatiramer Acetate (GA) Treatment in Patients Presenting a Clinically Isolated Syndrome (CIS) on Long-Term Progression of MS

A clinical trial was undertaken to assess, within the time frame of 5 years, the neuroprotective effect of early versus delayed treatment with GA as reflected by clinical and MRI parameters measuring the accumulated irreversible brain tissue damage.

### Methods

481 subjects between the ages of 18 and 45 years, with a single well-defined unifocal neurological event compatible with MS, and exhibiting at least 2 cerebral lesions highly suspicious of MS on the screening MRI measuring 6 mm or more in diameter, are included and randomized in equal numbers to receive 20 mg GA or placebo.

Following conversion to CDMS or after 3 years of treatment, whichever comes first, all subjects in the study are switched to active treatment. Subjects already on 20 mg GA continue with their active treatment while subjects on placebo are switched to 20 mg GA for total treatment duration of 60 months (5 years). Subjects are evaluated at study centers at baseline, at months 1, 3, and every 3 months thereafter. MRI evaluations of T1 and T2 variables are assessed at screening, baseline, at 3 months, and every 3 months thereafter until conversion to CDMS or up to 3 years. An additional MRI assessment is performed upon conversion to CDMS only if no MRI is performed within the previous month. MRI is then performed at the next scheduled visit and every 6 months thereafter. For subjects who do not convert after 3 years, MRI is performed every 6 months upon switching to active treatment.

Brain atrophy, as measured by the change in brain volume according to the Structural Image Evaluation of Normalized Atrophy (SIENA) technique is assessed at baseline, every 12 months and at conversion to CDMS.

The volume of black holes is assessed at baseline and at every 6 months.

The count of new T1-weighted hypointense lesions is assessed every 6 months.

Exploratory endpoints are defined to assess the neuroprotective effect as reflected by clinical and MRI parameters comparing the group originally assigned to GA treatment with that randomized to receive placebo treatment (delayed start of treatment with GA). The 5-year data cohort will be used for inference.

The list of exploratory endpoints is:
1) The time from randomization to conversion to CDMS during the 5-year period;
2) Proportion of patients who convert to CDMS during the 5-year treatment period;
3) The 5-year relapse rate; repeated measures analysis of the total number of new T2 lesions at each visit during the 5-year period;
4) Repeated measures analysis of the change from baseline to each visit in T2 lesions volume;
5) Brain atrophy: repeated measures of the change from baseline to each visit in brain volume;
6) Repeated measures analysis of the total number of new T1 gadolinium enhancing lesions at each visit during the 5-year period;
7) Repeated measures analysis of the change from baseline to each visit in T1 gadolinium enhancing lesions volume during the 5-year period;
8) Repeated measures analysis of the change from baseline to each visit in hypointense lesions volume in enhanced T1 weighted images (“black holes”) during the 5-year period;
9) Repeated measures analysis of the total number of new T1 hypointense lesions at each visit during the 5-year period;
10) Repeated measures of the change from baseline to each visit in the MSFC Score;
11) Repeated measures of the change from baseline to each visit in the EDSS Score;
12) The time from randomization to conversion to CDMS, either during the placebo-controlled period, or during the 5-year period, is also analyzed including baseline Anti-MOG and anti-MOP antibodies as binary covariate(s).

### Results

In early treatment group vs. delayed start of treatment with GA group: the time from randomization to conversion to CDMS during the 5-year period is increased; the proportion of patients who convert to CDMS during the 5-year treatment period is decreased; the 5-year relapse rate is decreased; the level of Brain Atrophy is reduced; the level of disability is reduced (as measured by EDSS Score).

### Conclusions

Early GA treatment confers significant neuroprotective effect as reflected by clinical and MRI parameters comparing the group originally assigned to GA treatment with that randomized to receive placebo treatment (delayed start of treatment with GA). These results show that early, pre-diagnosis i.e., pre-CDMS, GA treatment confers long-term benefits on MS symptoms and on the progression of disability.

### Discussion

The results described herein show that GA delays the development of Clinically Definite Multiple Sclerosis (CDMS) when administered to patients presenting a single, clinically isolated syndrome (CIS) suggestive of MS. MS is a progressive disease and a single CIS is the manifestation of a disease which began before occurrence of the single CIS. Thus, the single CIS is a useful point of reference in the clinical trials described, but is not the initiation of disease. There are known risk factors for MS and these include any one of a clinically isolated syndrome (CIS), a single attack suggestive of MS without a lesion, the presence of a lesion (in any of the CNS, PNS, or myelin sheath) without a clinical attack, environmental factors (16, 17, 18), genetics (19, 20) and immunological components (21, 22, 23).

The results herein show, therefore, that administration of GA to a subject having any of the known risk factors will delay the onset of clinically definite multiple sclerosis and will also retard long-term progression of multiple sclerosis and its symptoms. Early treatment with GA demonstrated protection against progression to CDMS. Therefore, the results show effectiveness of GA treatment of patients with a first clinical event suggestive of MS.

### REFERENCES

1. Poser C M. Paty D W, Scheinberg L, et al. “New Diagnostic Criteria for Multiple Sclerosis: Guidelines for Research Protocols”, Annals of Neurology, March 1983, 13 (3): 227-230.
2. Brex P A et al., “A longitudinal study of abnormalities on MRI and disability from multiple sclerosis”, N Engl J Med 2002 Jan. 17, 346(3):158-64
3. Frohman E M et al., “The utility of MRI in suspected MS: report of the Therapeutics and Technology Assessment Subcommittee of the American Academy of Neurology”, Neurology, 2003, September 9, 61(5):602-11
4. “What are the Types of Multiple Sclerosis?”, 2005, <imagines.com/multiple-sclerosis/types-of-ms/types-of-multiple-sclerosis.htm>
5. “Multiple sclerosis: its diagnosis, symptoms, types and stages”, 2003<www.albany.net/-tjc/multiple-sclerosis.html>
6. “Copaxone”, Physician's Desk Reference, 2005, Medical Economics Co., Inc., Montvale, N.J., 3115.
7. Johnson K P et al., “Copolymer 1 reduces relapse rate and improves disability in relapsing-remitting multiple sclerosis: results of a phase III multicenter, double-blind placebo-controlled trial. The Copolymer 1 Multiple Sclerosis Study Group”, Neurology, 1995, 45, 1268-1276.
8. Cohen JA et al., Rovaris, “9006 Study Group. Randomized, double-blind, dose-comparison study of glatiramer acetate in relapsing-remitting MS”, Neurology 2007, Mar. 20, 68(12):939-44.
9. Comi G et al., “European/Canadian Multicenter, Double-Blind, Randomized, Placebo-controlled study of the effects of Glatiramer acetate on magnetic resonance imaging-measured disease activity and burden in patients with relapsing multiple sclerosis”, Ann. Neurol., 49, 290-297, 2001.
10. Debra H., “Early, Aggressive Treatment Of Ms-Is It For Everyone?”, Clinical trends and news in neurology, 9, No. 8, 2001
11. DeNoon D J., “Caution Urged In Early MS Treatment-Too Soon For ‘Treat-All’ Approach To Multiple Sclerosis?” WebMD Medical News Aug. 2, 2007<www.medicinenet.com/script/main/art.asp?articlekey=83017
12. Pittock S J, at al., “Clinical implications of benign multiple sclerosis: a 20-year population-based follow-up study”, Ann Neurol. 2004 August, 56(2):303-6.
13. Poser C M and Brinar V V, “Diagnostic criteria for multiple sclerosis”, Clinical Neurology and Neurosurgery, 2001 April, 103(1):1-11
14. Smith S M, et al., “Accurate, robust and automated longitudinal and cross-sectional brain change analysis”, NeuroImage 2002, 17, 479-489.
15. World of MS<www.msif.org/en/aboutms/symptoms.html>
16. Marrie R A, “Environmental risk factors in multiple sclerosis aetiology”, Lancet Neurol. 2004 December, 3(12):709-18.
17. Ascherio A, Munger K L, “Environmental risk factors for multiple sclerosis. Part I: the role of infection”, Ann Neural, 2007 April, 61(4):288-99.
18. Ascherio A, Munger K L, “Environmental risk factors for multiple sclerosis. Part II: non-infectious factors”, Ann Neurol, 2007 July, 61(6):504-13.
19. Niino M, “Recent advances in genetic analysis of multiple sclerosis: genetic associations and therapeutic implications”, Expert Rev Neurother, 2007, September, 7(9):1175-88.
20. Reich D, “A whole-genome admixture scan finds a candidate locus for multiple sclerosis susceptibility”, Nat Genet, 2005, October, 37(10):1113-8, Epub 2005, September 25.
21. McFarland HE, “Multiple sclerosis: a complicated picture of autoimmunity”, Nat Immunol, 2007, September 8(9):913-9.
22. Lutterotti A, “Biological markers for multiple sclerosis”, Curr Med Chem, 2007, 14(18):1956-65.
23. Rinaldi L. and Gallo P., “Immunological markers in multiple sclerosis: tackling the missing elements”, Neural Sci, 2005, 26: 8215-5217.

What is claimed is:

1. A method for reducing the frequency of relapses in a patient who experienced a single clinical attack consistent with multiple sclerosis and who has at least one lesion consistent with multiple sclerosis prior to development of clinically definite multiple sclerosis (CDMS), the method consisting essentially of periodically administering to the patient a pharmaceutical composition comprising a pharmaceutically acceptable carrier and an amount of glatiramer acetate therapeutically effective to increase the time to relapse in the patient.

2. The method of claim 1, wherein the time to relapse is increased by 50%.

3. The method of claim 1, wherein the single clinical attack includes a clinical episode of optic neuritis, blurring of vision, diplopia, involuntary rapid eye movement, blindness, loss of balance, tremors, ataxia, vertigo, clumsiness of a limb, lack of co-ordination, weakness of one or more extremity, altered muscle tone, muscle stiffness, spasms, tingling, paraesthesia, burning sensations, muscle pains, facial pain, trigeminal neuralgia, stabbing sharp pains, burning tingling pain, slowing of speech, slurring of words, changes in rhythm of speech, dysphagia, fatigue, bladder problems, bowel problems, impotence, diminished sexual arousal, loss of sensation, sensitivity to heat, loss of short term memory, loss of concentration, or loss of judgment or reasoning.

4. The method of claim 1, wherein the at least one lesion is detectable by an MRI scan and is associated with brain tissue inflammation, myelin sheath damage or axonal damage.

5. The method of claim 4, wherein the lesion is a demyelinating white matter lesion visible on brain MRI.

6. The method of claim 5, wherein the white matter lesions are at least 3 mm in diameter.

7. The method of claim 1, wherein the periodic administration is once-a-day.

8. The method of claim 7, wherein the therapeutically effective amount of glatiramer acetate is 20 mg.

9. The method of claim 8, wherein the administration is subcutaneous.

10. The method of claim 9, wherein the amount of glatiramer acetate is subcutaneously administered via a prefilled syringe.

11. A method for delaying onset of clinically definite multiple sclerosis in a patient presenting a first clinical event consistent with multiple sclerosis and at least one lesion consistent with multiple sclerosis comprising periodically administering to the patient as monotherapy a pharmaceutical composition comprising a pharmaceutically acceptable carrier and an amount of glatiramer acetate therapeutically effective to delay onset of clinically definite multiple sclerosis.

12. The method of claim 11, wherein the at least one lesion is detectable by an MRI scan and is associated with brain tissue inflammation, myelin sheath damage or axonal damage.

13. The method of claim 12, wherein the lesion is a demyelinating white matter lesion visible on brain MRI.

14. The method of claim 13 wherein the white matter lesions are at least 3 mm in diameter.

15. The method of claim 11, wherein the periodic administration is once-a-day.

16. The method of claim 15, wherein the therapeutically effective amount of glatiramer acetate is 20 mg.

17. The method of claim 16, wherein the administration is subcutaneous.

18. The method of claim 17, wherein the amount of glatiramer acetate is subcutaneously administered via prefilled syringe.

19. A method of treating a patient who has experienced a first clinical episode and has MRI features consistent with multiple sclerosis consisting essentially of subcutaneously administering once a day to the patient prior to conversion to clinically definite multiple sclerosis a pharmaceutical composition comprising mannitol and 20 mg of glatiramer acetate.
